(19) United States
(12) Patent Application Publication
(10) Pub. No.: US 2009/0221570 A1
(43) Pub. Date: Sep. 3, 2009

(54) Uses of 2-Phenyl-Substituted Imidazotriazinone Derivatives for Treating Pulmonary Hypertension

(75) Inventors: Helmut Haning, Wuppertal (DE); Peter Serno, Bergisch Gladbach (DE); Erwin Bischoff, Wuppertal (DE); Ernst Ulbrich, Eltville (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Helthcare AG, Leverkusen (DE)

(21) Appl. No.: 11/918,312

(22) PCT Filed: Mar. 27, 2006
(86) PCT No.: PCT/EP2006/002774

§ 371 Date: Mar. 4, 2009

(30) Foreign Application Priority Data
Apr. 9, 2005 (DE) 102005016345.9

(51) Int. Cl. A61K 31/53; A61K 31/5377; A61K 31/519; A61K 31/4985

(52) U.S. Cl. 514/233.2; 514/243; 514/252.16; 514/250; 514/236.2

(57) ABSTRACT

The present invention relates to the use of PDE 5 inhibitors generally and in particular of known 2-phenyl-substituted imidazotriazinone derivatives for manufacturing medicaments for the treatment of pathological states which can be treated by raising cGMP levels in certain tissues, such as, for example, of, for example: primary pulmonary hypertension, secondary pulmonary hypertension, pulmonary arterial hypertension, portopulmonary hypertension, hepatopulmonary syndrome, pulmonary hypertension caused by medicaments (amphetamines), interstitial lung disease, pulmonary hypertension occurring with HIV, thromboembolic pulmonary hypertension, pulmonary hypertension in children and neonates, pulmonary hypertension induced by atmospheric hypoxia (altitude sickness), COPD, emphysema, chronic bronchial asthma, mucoviscidosis-related pulmonary hypertension, right heart failure, left heart failure and global failure, and which can be treated by raising cGMP levels in certain tissues, such as, for example, isolated systiolic hypertension (ISH) and hardening of blood vessels, specifically of arterial blood vessels, and combination of PDE 5 inhibitors generally and in particular of known 2-phenyl-substituted imidazotriazinone derivatives with further therapeutic agents in the said indications.

### BACKGROUND OF THE INVENTION

The present invention relates to the use of PDE 5 inhibitors generally and in particular of known 2-phenyl-substituted imidazotriazinone derivatives for manufacturing medicaments for the treatment of pathological states which hitherto have been regarded as being very difficult to treat.

The cyclic nucleotide cGMP (cyclic guanosine monophosphate) is one of the most important intracellular messengers and is metabolized by certain phosphodiesterases (PDEs), in particular the isoenzyme PDE 5, [Drugs Fut. 26, 153-162 (2001)]. PDE 5 occurs in particular in vascular smooth muscle cell tissue, and less in the kidney, lung and the blood platelets. Owing to their vasorelaxant effect, PDE 5 inhibitors are proposed for the treatment of angina and high blood pressure, but mainly for the treatment of erectile dysfunction.

WO 99/24433 describes 2-phenyl-substituted imidazotriazinones, their cGMP-PDE-inhibiting effect and their use for the treatment of vascular disorders, in particular for the treatment of erectile dysfunction. WO 02/089808 and WO 03/011262 disclose uses of 2-phenyl-substituted imidazotriazinones.

The literature at present describes 11 phosphodiesterases differing in specificity in relation to the cyclic nucleotides cAMP and cGMP [cf. Fawcett et al., Proc. Nat. Acad. Sci. 97 (7), 3072-3077 (2000)]. Cyclic guanosine 3′,5′-monophosphate-metabolizing phosphodiesterases (cGMP-PDEs) are PDE 1, 2, 5, 6, 9, 10 and 11. The mentioned 2-phenyl-substituted imidazotriazinones used according to the invention are potent inhibitors of phosphodiesterase 5. Differential expression of the phosphodiesterases in different cells, tissues and organs, as well as the differential subcellular localization of these enzymes, make it possible in conjunction with selective inhibitors to raise selectively the cGMP concentration in specific cells, tissues and organs and thus allow various cGMP-regulated processes to be addressed, so that PDE 5 inhibitors can be used therapeutically in a number of pathological states which can be influenced by raising the cGMP level.

### SUMMARY OF THE INVENTION

It has now been found that it is possible by increasing the cGMP levels in certain tissues to treat even pathological conditions which have to date been difficult to influence, such as, for example, primary pulmonary hypertension, secondary pulmonary hypertension, pulmonary arterial hypertension, portopulmonary hypertension, hepatopulmonary syndrome, pulmonary hypertension caused by medicaments such as amphetamines, interstitial lung disease, pulmonary hypertension occurring with HIV, thromboembolic pulmonary hypertension, pulmonary hypertension in children and neonates, pulmonary hypertension induced by atmospheric hypoxia (altitude sickness), COPD, emphysema, chronic asthma, bronchiale, mucoviscidosis-related pulmonary hypertension, right-heart failure, left-heart failure and global failure.

It has likewise been found that it is also possible by increasing cGMP levels in certain tissues to treat isolated systolic hypertension (ISH) and the hardening of blood vessels, specifically arterial blood vessels, e.g. the aorta.

PDE 5 inhibitors preferred in this connection are those which, in the assay detailed hereinafter, inhibit PDE 5 with an IC₅₀ of less than 1 μM, preferably of less than 0.1 μM. The PDE 5 inhibitors used according to the invention are preferably also selective in relation to cAMP PDEs, in particular in relation to PDE 4. It is particularly preferred for the inhibition of PDE 5 to be at least ten times greater.

The PDE 5 inhibitors can be used to treat said diseases alone or in a combination according to the invention with other therapeutic agents. Therapeutic agents suitable for combination in this connection are:
inhalation of oxygen or NO
diuretics, antiarrhythmics, calcium channel blockers, vasodilators
prostanoids and derivatives, prostacyclin and its analogs, epoprostenol, beraprost, iloprost, treprostinil sodium, and endothelin receptor antagonists such as, for example, bosentan, and adrenomedullin
anticoagulants, further phosphodiesterase inhibitors, including those which relax smooth muscles via increasing cAMP, such as, for example, the PDE3 inhibitor milrinone
cardiac glycosides
beta-receptor blockers
alpha-receptor blockers
ACE inhibitors
angiotensin II receptor antagonists
nitrates, molsidomine

The medicaments can be supplied by the routes known hitherto, depending on the pathological condition and, in the case of a combination, on the pharmacokinetics of the combination partners. Oral, intravenous and inhalational administration are particularly preferred in this connection.

Suitable for this purpose are on the one hand fast-release dosage forms such as coated or uncoated tablets, capsules, powders, granules, pharmaceutical forms which disintegrate in the mouth, oral solutions, solutions for injection, solutions for infusion, solutions for inhalation, suspensions for inhalation or powders for inhalation. Controlled release formulations are particularly preferred in each case for the oral administration of PDE 5 inhibitors or further therapeutic agent having a short half-life.

### DETAILED DESCRIPTION OF THE INVENTION AND PREFERRED EMBODIMENTS

Compounds having an inhibitory effect on cGMP PDEs are described for example in the following publications: EP-A-0 201 188, EP-A-0 214 708, EP-A-0 293 063, EP-A-0 319 050, EP-A-0 347 027, EP-A-0 347 146, EP-A-0 349 239, EP-A-0 351 058, EP-A-0 352 960, EP-A-0 371 731, EP-A-0 395 328, EP-A-0 400 799, EP-A-0 428 268, EP-A-0 442 204, EP-A-0 463 756, EP-A-0 526 004, EP-A-0 579 496, EP-A-0 607 439, EP-A-0 640 599, EP-A-0 669 324, EP-A-0 686 625, EP-A-0 722 936, U.S. Pat. No. 4,060,615, U.S. Pat. No. 5,294,612, WO 91/19717, WO 94/19351, WO 94/22855, WO 96/32379, WO 97/03070, JP-A-05222000 (CAPLUS 1994, 191719).

Compounds having an inhibitory effect on the cGMP-specific PDE (corresponds to PDE 5) are described for example in the following publications: EP-A-0 636 626, EP-A-0 668 280, EP-A-0 722 937, EP-A-0 722 943, EP-A-0 722 944, EP-A-0 758 653, EP-A-0 995 750, EP-A-0 995 751, EPA-1 092 719, WO 94/28902, WO 95/19978, WO 96/16657, WO 96/28159, WO 96/28429, WO 98/49166, WO 99/24433, WO 99/67244, WO 00/78767, WO 01/12608, WO 01/18004, WO 01/19369, WO 01/19802, WO 01/21620, WO 01/27105, J. Med. Chem. 39, 1635-1644 (1996), J. Med. Chem. 43, 1257-1263 (2000), Drugs Fut. 26, 153-162 (2001).

The disclosure of these publications, in particular the compounds disclosed therein, is incorporated herein by reference.

One aspect of the present invention relates to the use of these cGMP PDE inhibitors and more particularly of compounds of the general formula (I)

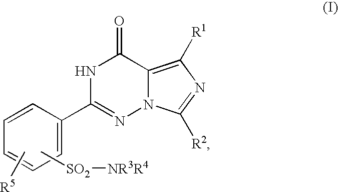

in which
R¹ is methyl or ethyl,
R² is ethyl or propyl,
R³ and R⁴ are identical or different and are a straight-chain or branched alkyl chain having up to 5 carbon atoms which is optionally substituted up to twice identically or differently by hydroxy or methoxy,
or
R³ and R⁴ together with the nitrogen atom form a piperidinyl, morpholinyl, thiomorpholinyl ring or a radical of the formula

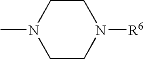

in which
R⁶ is hydrogen, formyl, acyl or alkoxycarbonyl having in each case up to 3 carbon atoms,
   or
   is straight-chain or branched alkyl having up to 3 carbon atoms which is optionally substituted once to twice, identically or differently, by hydroxy, carboxyl, straight-chain or branched alkoxy or alkoxycarbonyl having in each case up to 3 carbon atoms or by groups of the formulae —(CO)_{f}—NR⁷R⁸ or —P(O)(OR⁹)(OR¹⁰),
   in which
   f is a number 0 or 1,
   R⁷ and R⁸ are identical or different and are hydrogen or methyl,
   R⁹ and R¹⁰ are identical or different and are hydrogen, methyl or ethyl,
or
R⁶ is cyclopentyl,
and the heterocycles mentioned under R³ and R⁴ and formed together with the nitrogen atom are optionally substituted once to twice, identically or differently, optionally also geminally, by hydroxy, formyl, carboxyl, acyl or alkoxycarbonyl having in each case up to 3 carbon atoms or groups of the formulae —P(O)(OR¹¹)(OR¹²) or —(CO)ᵢ—NR¹³R¹⁴,
   in which
   R¹¹ and R¹² are identical or different and are hydrogen, methyl or ethyl,
   i is a number 0 or 1,
   and
   R¹³ and R¹⁴ are identical or different and are hydrogen or methyl,
and/or the heterocycles mentioned under R³ and R⁴ and formed together with the nitrogen atom are optionally substituted by straight-chain or branched alkyl having up to 3 carbon atoms, which is optionally substituted once to twice, identically or differently, by hydroxy, carboxyl or by a radical of the formula —P(O)OR¹⁵OR¹⁶,
   in which
   R¹⁵ and R¹⁶ are identical or different and are hydrogen, methyl or ethyl,
and/or the heterocycles mentioned under R³ and R⁴ and formed together with the nitrogen atom are optionally substituted by N-linked piperidinyl or pyrrolidinyl,
and
R⁵ is ethoxy or propoxy,
and the salts and solvates thereof and the solvates of the salts,
   for the treatment of for example primary pulmonary hypertension, secondary pulmonary hypertension, pulmonary arterial hypertension, portopulmonary hypertension, hepatopulmonary syndrome, pulmonary hypertension caused by medicaments (amphetamines), interstitial lung disease, pulmonary hypertension occurring with HIV, thromboembolic pulmonary hypertension, pulmonary hypertension in children and neonates, pulmonary hypertension induced by atmospheric hypoxia (altitude sickness), COPD, emphysema, chronic bronchial asthma, mucoviscidosis-related pulmonary hypertension, right-heart failure, left-heart failure and global failure, and combination of PDE 5 inhibitors in general and in particular of known 2-phenyl-substituted imidazotriazinone derivatives with standard therapeutic agents in the stated indications and
   for the treatment of isolated systolic hypertension (ISH) and the hardening of blood vessels, specifically arterial blood vessels, e.g. the aorta.

The compounds used according to the invention may, depending on their structure, exist in stereoisomeric forms (enantiomers, diastereomers). The invention therefore includes the use of the enantiomers and diastereomers and respective mixtures thereof. The stereoisomerically pure constituents can be isolated from such mixtures of enantiomers and/or diastereomers in a known manner.

If the compounds used according to the invention can occur in tautomeric forms, the present invention includes all tautomeric forms.

Salts preferred in the context of the present invention are physiologically acceptable salts of the compounds used according to the invention. Also included are salts which are themselves unsuitable for pharmaceutical applications but can be used for example to isolate or purify the compounds used according to the invention.

Physiologically acceptable salts of the compounds used according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, ethanedisulfonic acid, 2-hydroxyethanesulfonic acid, toluenesulfonic acid, benzenesulfonic acid, naphthalenesulfonic acid, naphthalenedisulfonic acid, adipic acid, ascorbic acid, succinic acid, acetic acid, trifluoroacetic acid, propionic acid, lactic acid as (+)-L-lactic acid or racemic (±)-DL-lactic acid, malonic acid, tartaric acid, malic acid, citric acid, fumaric acid, maleic acid, benzoic acid, gluconic acid, glucuronic acid, lactobionic acid, nicotinic acid, pamoic acid and cation exchanger resins.

Physiologically acceptable salts of the compounds used according to the invention also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refer in the context of the invention to those forms of the compounds used according to the invention which form in the solid or liquid state a complex by coordination with solvent molecules. Hydrates are a specific type of solvates in which the coordination takes place with water. Hydrates are preferred as solvates in the context of the present invention. Hydrates can be prepared for example by crystallizing the relevant compound from water or a hydrous solvent.

The present invention also includes in addition prodrugs of the compounds used according to the invention. The term “prodrugs” includes compounds which themselves may be biologically active or inactive but are converted (for example by metabolism or hydrolysis) during their residence time in the body into the compounds used according to the invention.

In the context of the present invention, the substituents have the following meaning unless specified otherwise:

An acyl radical having 1 to 3 carbon atoms is in the context of the invention for example formyl, acetyl or propionyl.

A straight-chain or branched alkoxy radical having 1 to 3 carbon atoms is in the context of the invention for example methoxy, ethoxy, n-propoxy or isopropoxy.

An alkoxycarbonyl radical having 1 to 3 carbon atoms is in the context of the invention for example methoxycarbonyl, ethoxycarbonyl or propoxycarbonyl.

A straight-chain or branched alkyl radical having 1 to 5 or 1 to 3 carbon atoms is in the context of the invention for example methyl, ethyl, n-propyl, isopropyl, tert-butyl or n-pentyl. Straight-chain or branched alkyl radicals having 1 to 4 or 1 to 3 carbon atoms are preferred.

A further embodiment of the invention relates to the use according to the invention of compounds of the general formula (I) in which the radicals R⁵ and —SO₂NR³R⁴ are positioned parallel to one another on the phenyl ring, and R¹, R², R³, R⁴ and R⁵ each have the meaning indicated above.

A further embodiment of the invention relates to the use according to the invention of compounds of the general formula (Ia)

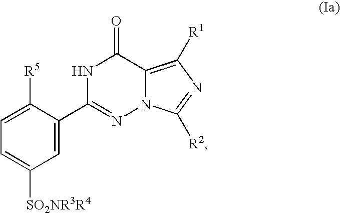

in which R¹, R², R³, R⁴ and R⁵ each have the meaning indicated above,
and the salts and solvates thereof and the solvates of the salts.

The use according to the invention of the following compounds is preferred:
2-[2-Ethoxy-5-(4-methylpiperazine-1-sulfonyl)-phenyl]-5,7-dimethyl-3H-imidazo[5,1-f][1,2,4]-triazin-4-one;
2-[2-Ethoxy-5-(4-hydroxyethylpiperazine-1-sulfonyl)-phenyl]-5,7-dimethyl-3H-imidazo[5,1-f]-[1,2,4]triazin-4-one;
2-[2-Ethoxy-5-(4-hydroxypiperidine-1-sulfonyl)-phenyl]-5,7-dimethyl-3H-imidazo[5,1-f][1,2,4]-triazin-4-one;
2-[2-Ethoxy-5-(4-hydroxymethylpiperidine-1-sulfonyl)-phenyl]-5,7-dimethyl-3H-imidazo[5,1-f]-[1,2,4]triazin-4-one;
2-[2-Ethoxy-5-(3-hydroxypyrrolidine-1-sulfonyl)-phenyl]-5,7-dimethyl-3H-imidazo[5,1-f][1,2,4]-triazin-4-one;
4-Ethoxy-N-ethyl-N-(2-hydroxyethyl)-3-(5,7-dimethyl-4-oxo-3,4-dihydro-imidazo[5,1-f][1,2,4]-triazin-2-yl)benzenesulfonamide;
N,N-Diethyl-4-ethoxy-3-(5,7-dimethyl-4-oxo-3,4-dihydroimidazo[5,1-f][1,2,4]triazin-2-yl)-benzenesulfonamide;
2-[2-Ethoxy-5-(4-(2-pyrimidinyl)-piperazine-1-sulfonyl)-phenyl]-5,7-dimethyl-3H-imidazo-[5,1-f]-[1,2,4]triazin-4-one;
2-[2-Ethoxy-5-(morpholine-4-sulfonyl)-phenyl]-5,7-dimethyl-3H-imidazo[5,1-f][1,2,4]triazin-4-one;
2-[2-Ethoxy-5-(1,4-dioxa-6-azaspiro[4.4]nonane-6-sulfonyl)-phenyl]-5,7-dimethyl-3H-imidazo[5,1-f][1,2,4]triazin-4-one;
N,N-Bis-(2-Methoxyethyl)-4-ethoxy-3-(5,7-dimethyl-4-oxo-3,4-dihydroimidazo[5,1-f][1,2,4]-triazin-2-yl)-benzenesulfonamide;
N-(3-Isoxazolyl)-4-ethoxy-3-(5,7-dimethyl-4-oxo-3,4-dihydroimidazo[5,1-f][1,2,4]triazin-2-yl)-benzenesulfonamide;
2-[2-Ethoxy-5-(2-tert.-butoxycarbonylaminomethylmorpholine-4-sulfonyl)-phenyl]-5,7-dimethyl-3H-imidazo[5,1-f][1,2,4]triazin-4-one;
2-[2-Ethoxy-5-(4-phenylpiperazine-1-sulfonyl)-phenyl]-5,7-dimethyl-3H-imidazo[5,1-f][1,2,4]-triazin-4-one;
2-[2-Ethoxy-5-(3-hydroxy-3-methoxymethylpyrrolidine-1-sulfonyl)-phenyl]-5,7-dimethyl-3H-imidazo[5,1-f][1,2,4]triazin-4-one;
2-[2-Ethoxy-5-(4-methylpiperazine-1-sulfonyl)-phenyl]-5-methyl-7-propyl-3H-imidazo[5,1-]-[1,2,4]triazin-4-one;
2-[2-Ethoxy-5-(4-methylpiperazine-1-sulfonyl)-phenyl]-5-methyl-7-propyl-3H-imidazo[5,1-f]-[1,2,4]triazin-4-one lactate;
2-[2-Ethoxy-5-(4-methylpiperazine-1-sulfonyl)-phenyl]-5-methyl-7-propyl-3H-imidazo[5,1-f]-[1,2,4]triazin-4-one hydrochloride;
2-[2-Ethoxy-5-(4-ethylpiperazine-1-sulfonyl)-phenyl]-5-methyl-7-propyl-3H-imidazo[5,1-f][1,2,4]-triazin-4-one;
2-[2-Ethoxy-5-(4-ethylpiperazine-1-sulfonyl)-phenyl]-5-methyl-7-propyl-3H-imidazo[5,1-f][1,2,4]-triazin-4-one hydrochloride;
2-[2-Ethoxy-5-(4-methyl-1-amino-piperazine-1-sulfonyl)-phenyl]-5-methyl-7-propyl-3H-imidazo-[5,1-f][1,2,4]triazin-4-one;
2-[2-Ethoxy-5-(4-hydroxyethyl-1-amino-piperazine-1-sulfonyl)-phenyl]-5-methyl-7-propyl-3H-imidazo[5,1-f][1,2,4]triazin-4-one;
N,N-Bishydroxyethylaminoethyl-4-ethoxy-3-(5-methyl-4-oxo-7-propyl-3,4-dihydro-imidazo[5,1-f]-[1,2,4]triazin-2-yl)benzenesulfonamide;
2-[2-Ethoxy-5-(4-dimethoxyphosphorylmethyl-piperazine-1-sulfonyl)-phenyl]-5-methyl-7-propyl-3H-imidazo[5,1-f][1,2,4]triazin-4-one;
2-[2-Ethoxy-5-(4-diethoxyphosphorylmethyl-piperidine-1-sulfonyl)-phenyl]-5-methyl-7-propyl-3H-imidazo[5,1-f][1,2,4]triazin-4-one;
2-[2-Ethoxy-5-(4-hydroxypiperidine-1-sulfonyl)-phenyl]-5-methyl-7-propyl-3H-imidazo[5,1-f]-[1,2,4]triazin-4-one;
2-{2-Ethoxy-5-[4-(2-hydroxyethyl)-piperazine-1-sulfonyl]-phenyl}-5-methyl-7-propyl-3H-imidazo[5,1-f][1,2,4]triazin-4-one;
2-{2-Ethoxy-5-[4-(2-hydroxyethyl)-piperazine-1-sulfonyl]-phenyl}-5-methyl-7-propyl-3H-imidazo[5,1-f][1,2,4]triazin-4-one hydrochloride;
2-{2-Ethoxy-5-[4-(3-hydroxypropyl)-piperazine-1-sulfonyl]-phenyl}-5-methyl-7-propyl-3H-imidazo[5,1-f][1,2,4]triazin-4-one;
N-Allyl-4-ethoxy-N-(2-hydroxyethyl)-3-(5-methyl-4-oxo-7-propyl-3,4-dihydro-imidazo[5,1-f]-[1,2,4]triazin-2-yl)benzenesulfonamide;
N-Ethyl-4-ethoxy-N-(2-hydroxyethyl)-3-(5-methyl-4-oxo-7-propyl-3,4-dihydro-imidazo[5,1-f]-[1,2,4]triazin-2-yl)benzenesulfonamide;
N,N-Diethyl-4-ethoxy-3-(5-methyl-4-oxo-7-propyl-3,4-dihydro-imidazo[5,1-f]-[1,2,4]triazin-2-yl)-benzenesulfonamide;
N-(2-Methoxyethyl)-3-(5-methyl-4-oxo-7-propyl-3,4-dihydro-imidazo[5,1-f][1,2,4]triazin-2-yl)-4-ethoxy-benzenesulfonamide;
N-(2-N,N-Dimethylethyl)-3-(5-methyl-4-oxo-7-propyl-3,4-dihydro-imidazo[5,1-f][1,2,4]triazin-2-yl)-4-ethoxy-benzenesulfonamide;
N-[3-(1-Morpholino)propyl]-3-(5-methyl-4-oxo-7-propyl-3,4-dihydro-imidazo[5,1-f][1,2,4]triazin-2-yl)-4-ethoxy-benzenesulfonamide;
N-{3-[1-(4-Methyl)piperazino]-propyl}-3-(5-methyl-4-oxo-7-propyl-3,4-dihydro-imidazo[5,1-f]-[1,2,4]triazin-2-yl)-4-ethoxy-benzenesulfonamide;
2-{2-Ethoxy-5-[4-(2-methoxyethyl)-piperazine-1-sulfonyl]-phenyl}-5-methyl-7-propyl-3H-imidazo[5,1-f][1,2,4]triazin-4-one;
2-{2-Ethoxy-5-[4-(2-N,N-dimethyl-ethyl)-piperazine-1-sulfonyl]-phenyl}-5-methyl-7-propyl-3H-imidazo[5,1-f][1,2,4]triazin-4-one;
2-{2-Ethoxy-5-[4-(3-N,N-dimethyl-propyl)-piperazine-1-sulfonyl]-phenyl}-5-methyl-7-propyl-3H-imidazo[5,1-f][1,2,4]triazin-4-one;
2-[2-Ethoxy-5-(4-dioxolano-piperidine-1-sulfonyl)-phenyl]-5-methyl-7-propyl-3H-imidazo[5,1-f]-[1,2,4]triazin-4-one;
2-[2-Ethoxy-5-(4-(5-methyl-4-furoxancarbonyl)-piperazine-1-sulfonyl)-phenyl]-5-methyl-7-propyl-3H-imidazo[5,1-f][1,2,4]triazin-4-one;
2-{2-Ethoxy-5-[4-acetyl-piperazine-1-sulfonyl]-phenyl}-5-methyl-7-propyl-3H-imidazo[5,1-f]-[1,2,4]triazin-4-one;
2-{2-Ethoxy-5-[4-formyl-piperazine-1-sulfonyl]-phenyl}-5-methyl-7-propyl-3H-imidazo[5,1-f]-[1,2,4]triazin-4-one;
2-[2-Ethoxy-5-(3-butylsydnonimine)-1-sulfonyl)-phenyl]-5-methyl-7-propyl-3H-imidazo[5,1-f]-[1,2,4]triazin-4-one;
5-Methyl-2-[5-(4-methyl-piperazine-1-sulfonyl)-2-propoxy-phenyl]-7-propyl-3H-imidazo[5,1-f]-[1,2,4]triazin-4-one;
5-Methyl-2-[5-(4-methyl-piperazine-1-sulfonyl)-2-propoxy-phenyl]-7-propyl-3H-imidazo[5,1-f]-[1,2,4]triazin-4-one hydrochloride;
2-[5-(4-Hydroxypiperidine-1-sulfonyl)-2-propoxy-phenyl]-5-methyl-7-propyl-3H-imidazo[5,1-f]-[1,2,4]triazin-4-one;
2-[5-(4-Hydroxymethylpiperidine-1-sulfonyl)-2-propoxy-phenyl]-5-methyl-7-propyl-3H-imidazo-[5,1-f][1,2,4]triazin-4-one;
2-{5-[4-(2-Hydroxyethyl)-piperazine-1-sulfonyl]-2-propoxy-phenyl}-5-methyl-7-propyl-3H-imidazo[5,1-f][1,2,4]triazin-4-one;
N-(1,1-Dioxotetrahydro-1λ⁶-thiophen-3-yl)-3-(5-methyl-4-oxo-7-propyl-3,4-dihydro-imidazo-[5,1-f][1,2,4]triazin-2-yl)-4-propoxy-benzenesulfonamide;
N-(2-Dimethylaminoethyl)-N-methyl-3-(5-methyl-4-oxo-7-propyl-3,4-dihydro-imidazo[5,1-f]-[1,2,4]triazin-2-yl)-4-propoxy-benzenesulfonamide;
3-(5-Methyl-4-oxo-7-propyl-3,4-dihydro-imidazo[5,1-f][1,2,4]triazin-2-yl)-N-(3-morpholin-4-yl-propyl)-4-propoxy-benzenesulfonamide;
N,N-Bis-(2-hydroxyethyl)-3-(5-methyl-4-oxo-7-propyl-3,4-dihydro-imidazo[5,1-f][1,2,4]triazin-2-yl)-4-propoxy-benzenesulfonamide;
N-(3-Hydroxybenzyl)-3-(5-methyl-4-oxo-7-propyl-3,4-dihydro-imidazo[5,1-f][1,2,4]triazin-2-yl)-4-propoxy-benzenesulfonamide;
N-Ethyl-N-(2-hydroxyethyl)-3-(5-methyl-4-oxo-7-propyl-3,4-dihydro-imidazo[5,1-f][1,2,4]triazin-2-yl)-4-propoxy-benzenesulfonamide;
N-(3-Ethoxypropyl)-3-(5-methyl-4-oxo-7-propyl-3,4-dihydro-imidazo[5,1-f][1,2,4]triazin-2-yl)-4-propoxy-benzenesulfonamide;
2-[5-(4-Hydroxypiperidine-1-sulfonyl)-2-propoxy-phenyl]-5-methyl-7-propyl-3H-imidazo[5,1-f]-[1,2,4]triazin-4-one;
3-(5-Methyl-4-oxo-7-propyl-3,4-dihydro-imidazo[5,1-f][1,2,4]triazin-2-yl)-4-propoxy-N-pyridin-4-yl-benzenesulfonamide;
N,N-Diethyl-3-(5-methyl-4-oxo-7-propyl-3,4-dihydro-imidazo[5,1-f][1,2,4]triazin-2-yl)-4-propoxy-benzenesulfonamide;
1-[3-(5-Methyl-4-oxo-7-propyl-3,4-dihydro-imidazo[5,1-f][1,2,4]triazin-2-yl)-4-propoxy-benzene-sulfonyl]piperidine-4-carboxylic acid;
5-Methyl-2-[5-(morpholine-4-sulfonyl)-2-propoxy-phenyl]-7-propyl-3H-imidazo[5,1-f][1,2,4]-triazin-4-one;
N-(2-Hydroxyethyl)-N-methyl-3-(5-methyl-4-oxo-7-propyl-3,4-dihydro-imidazo[5,1-f][1,2,4]-triazin-2-yl)-4-propoxy-benzenesulfonamide;
N-(2-Hydroxyethyl)-3-(5-methyl-4-oxo-7-propyl-3,4-dihydro-imidazo[5,1-f][1,2,4]triazin-2-yl)-4-propoxy-N-propyl-benzenesulfonamide;
N-[2-(3,4-Dimethoxyphenyl)-ethyl]-N-methyl-3-(5-methyl-4-oxo-7-propyl-3,4-dihydro-imidazo-[5,1-f][1,2,4]triazin-2-yl)-4-propoxy-benzenesulfonamide;
N-Allyl-N-(2-hydroxyethyl)-3-(5-methyl-4-oxo-7-propyl-3,4-dihydro-imidazo[5,1-f][1,2,4]triazin-2-yl)-4-propoxybenzenesulfonamide;
N-Ally-N-cyclopentyl-3-(5-methyl-4-oxo-7-propyl-3,4-dihydro-imidazo[5,1-f]-[1,2,4]triazin-2-yl)-4-propoxybenzenesulfonamide;
N-Allyl-N-ethyl-3-(5-methyl-4-oxo-7-propyl-3,4-dihydro-imidazo[5,1-f][1,2,4]triazin-2-yl)-4-propoxybenzenesulfonamide;
2-[2-Ethoxy-4-methoxy-5-(4-methylpiperazine-1-sulfonyl)-phenyl]-5-methyl-7-propyl-3H-imidazo-[5,1-f][1,2,4]triazin-4-one;
2-{2-Ethoxy-5-[4-(2-hydroxyethyl)-piperazine-1-sulfonyl]-4-methoxy-phenyl}-5-methyl-7-propyl-3H-imidazo[5,1-f][1,2,4]triazin-4-one;
4-Ethoxy-N-ethyl-N-(2-hydroxyethyl)-2-methoxy-5-(5-methyl-4-oxo-7-propyl-3,4-dihydro-imidazo[5,1-f][1,2,4]triazin-2-yl)-benzenesulfonamide;
4-Ethoxy-N-(4-ethoxyphenyl)-2-methoxy-5-(5-methyl-4-oxo-7-propyl-3,4-dihydro-imidazo[5,1-f]-[1,2,4]triazin-2-yl)-benzenesulfonamide;
4-Ethoxy-N-ethyl-N-(2-hydroxyethyl)-3-(5-ethyl-4-oxo-7-propyl-3,4-dihydroimidazo[5,1-f][1,2,4]-triazin-2-yl)benzenesulfonamide;
N-(2-Methoxyethyl)-3-(5-ethyl-4-oxo-7-propyl-3,4-dihydro-imidazo[5,1-f][1,2,4]triazin-2-yl)-4-ethoxybenzenesulfonamide;
N,N-Bis-(2-methoxyethyl)-3-(5-ethyl-4-oxo-7-propyl-3,4-dihydroimidazo[5,1-f][1,2,4]triazin-2-yl)-4-ethoxybenzenesulfonamide;
2-[5-(4-Hydroxypiperidine-1-sulfonyl)-2-ethoxyphenyl]-5-ethyl-7-propyl-3H-imidazo[5,1-f][1,2,4]triazin-4-one;
2-[5-(4-Hydroxymethylpiperidine-1-sulfonyl)-2-ethoxy-phenyl]-5-ethyl-7-propyl-3H-imidazo[5,1-f][1,2,4]triazin-4-one;
2-{2-Ethoxy-5-[4-(2-hydroxyethyl)-piperazine-1-sulfonyl]-phenyl}-5-ethyl-7-propyl-3H-imidazo-[5,1-f][1,2,4]triazin-4-one;
2-[2-Ethoxy-5-(4-methylpiperazine-1-sulfonyl)-phenyl]-5-ethyl-7-propyl-3H-imidazo[5,1-f][1,2,4]-triazin-4-one;
2-[2-Ethoxy-5-(4-methylpiperazine-1-sulfonyl)-phenyl]-5-ethyl-7-propyl-3H-imidazo[5,1-f][1,2,4]-triazin-4-one hydrochloride;
3-(5-Ethyl-4-oxo-7-propyl-3,4-dihydro-imidazo[5,1-f][1,2,4]triazin-2-yl)-N-(3-morpholin-4-yl-propyl)-4-ethoxybenzenesulfonamide;
N-(2-Hydroxyethyl)-3-(5-ethyl-4-oxo-7-propyl-3,4-dihydro-imidazo[5,1-f][1,2,4]triazin-2-yl)-4-ethoxy-N-propyl-benzenesulfonamide;
2-[2-Ethoxy-5-(4-ethyl-piperazine-1-sulfonyl)-phenyl]-5-methyl-7-propyl-3H-imidazol[5,1-f]-[1,2,4]triazin-4-one hydrochloride trihydrate;
2-[2-Ethoxy-5-(4-ethyl-piperazine-1-sulfonyl)-phenyl]-5-methyl-7-propyl-3H-imidazo[5,1-f]-[1,2,4]triazin-4-one dihydrochloride.

Compounds particularly preferably used are listed in table A:

TABLE A
Structure

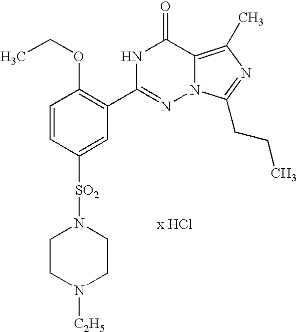

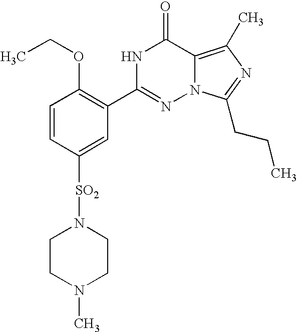

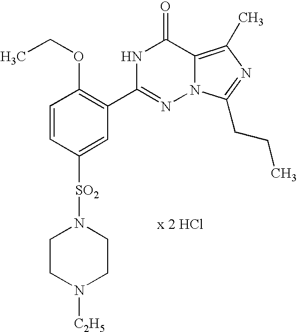

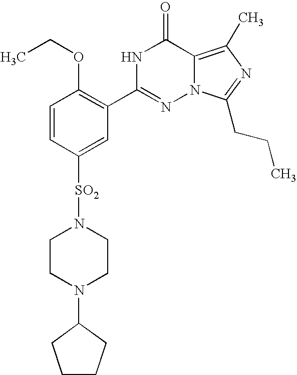

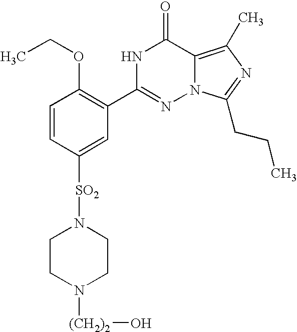

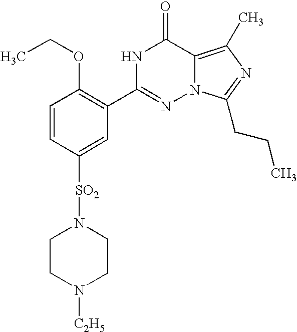

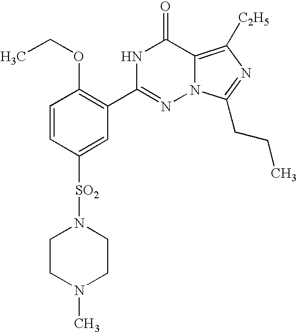

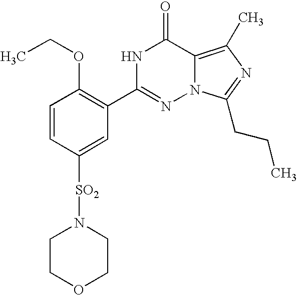

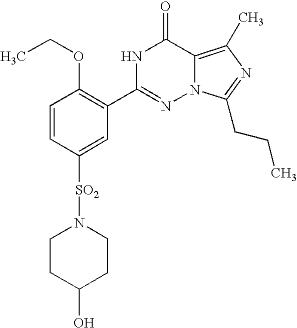

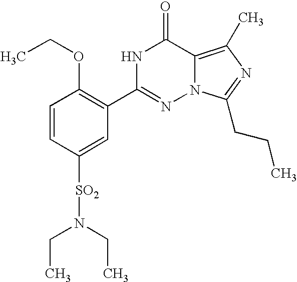

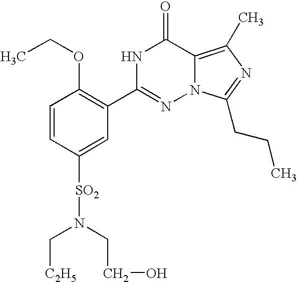

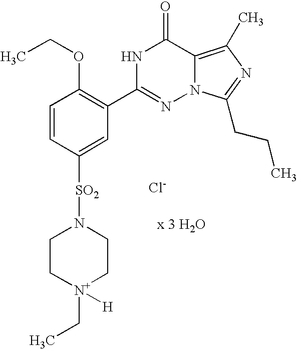

The compounds of the formulae (I) and (Ia) and of table A used according to the invention, and their preparation are described in WO 99/24433. The disclosure of WO 99/24433 is incorporated herein by reference.

A further embodiment of the invention relates to the use of the compounds of the general formulae (I) and (Ia) for manufacturing a medicament for the treatment of, for example, primary pulmonary hypertension, secondary pulmonary hypertension, pulmonary arterial hypertension, portopulmonary hypertension, hepatopulmonary syndrome, pulmonary hypertension caused by medicaments (amphetamines), interstitial lung disease, pulmonary hypertension occurring with HIV, thromboembolic pulmonary hypertension, pulmonary hypertension in children and neonates, pulmonary hypertension induced by atmospheric hypoxia (altitude sickness), COPD, emphysema, chronic bronchial asthma, mucoviscidosis-related pulmonary hypertension, right-heart failure, left-heart failure and global failure, and combination of PDE 5 inhibitors in general and in particular of known 2-phenyl-substituted imidazotriazinone derivatives with standard therapeutic agents in the stated indications.

A further aspect of the present invention is the use of sildenafil and its salts, e.g. the citrate, also in various modifications, for the treatment of isolated systolic hypertension (ISH) and the hardening of blood vessels, specifically arterial blood vessels, e.g. the aorta. A further aspect of the present invention is the use of tadalafill and its modifications modifications for the treatment of isolated systolic hypertension (ISH) and the hardening of blood vessels, specifically arterial blood vessels, e.g. the aorta.

The present invention further relates to the use of the compounds of the invention in combination with one or more further therapeutic agents for the treatment of the abovementioned pathological states. A free combination is possible in this connection, with the cGMP PDE inhibitor and the further therapeutic agent(s) being administered in separate pharmaceutical forms. Administration of the combination partners at separate times is also possible. Alternatively, administration takes place together as fixed combination in which the combination partners form part of a pharmaceutical form or are present closely together in a joint pack.

A therapeutic agent suitable for combination is on the one hand the inhalation of oxygen or NO, it being possible for the inhalation also to be in gradually increasing or decreasing doses and pulsatile.

Further therapeutic agents suitable for combination are diuretics, antiarrhythmics, calcium channel blockers and vasodilators. Suitable as diuretic are for example hydrochlorothiazide, furosemide, piretamide, torasemide, potassium canrenoate or spironolactone. Antiarrhythmics which can be used are for example quinidine bisulfatetetrahydrate, disopyramide phosphate, ajmaline, prajmalium bitartrate, lidocain, mexiletine HCl, propafenone HCl, flecamide acetate, amiodarone HCl, sotalol HCl, ipratropium bromide or adenosine. The antiarrhythmics can moreover be administered orally or, as in the example of ajmaline, lidocain or adenosine, intravenously. Examples of suitable calcium channel blockers are nitrendipine, isradipine, felodipine, nilvadipine, nifedipine, nisoldipine, lacidipine, lercanidipine HCl, manidipine 2 HCl, nicardipine HCl or amlodipine, for example amlodipine maleate, amlodipine mesilate hydrate or amlodipine besilate. Examples of suitable vasodilators are sodium nitroprusside dihydrate, minoxidil or dihydralazine, for example as dihydralazine sulfate or dihydralazine mesilate.

Other combinable therapeutic agents are prostanoids such as, for example, PGI2 and derivatives, prostacyclin and its analogs, epoprostenol, beraprost, iloprost, treprostinil sodium, endothelin receptor antagonists such as, for example, bosentane, and adrenomedullin. Depending on the pharmacokinetic properties, the therapeutic agents to be combined are also supplied by inhalation or parenterally.

Other combinable therapeutic agents are anticoagulants such as warfarin, and further phosphodiesterase inhibitors, for example PDE3 inhibitors, PDE4 inhibitors, PDE5 inhibitors; for example from WO 2004022557, WO 2004019945, WO 2004018457, WO 2004018450, WO 2004018449, WO 2004017974, WO 2003074055, WO 2003070279, WO 2002085906, WO 2002085885, WO 2004018465, sildenafil or tadalafil.

Further combinable therapeutic agents are cardiac glycosides, for example digoxin, acetyldigoxin, metildigoxin or digitoxin.

Further combinable therapeutic agents are beta-blockers such as atenolol, propanolol, pindolol, bisoprolol, metoprolol. Also possible are celiprolol, talinolol, acebutolol HCl, oxprolol, nadolol, penbutolol sulfate, carteolol HCl, bupranolol HCl or mepindolo sulfate.

Further combinable therapeutic agents are alpha-receptor blockers such as, for example, doxazosin, prazosin or terazosin.

Further combinable therapeutic agents are ACE inhibitors such as, for example, enalapril maleate, captopril, lisinopril dihydrate, quinapril HCl, fosinopril sodium, trandolapril, benazepril HCl, ramipril or cilazapril.

Further combinable therapeutic agents are angiotensin II receptor antagonists such as losartan potassium, irbesartan, valsartan, candesartan cilexetil, eprosartan, telmisartan or olmesartan medoxomil.

Further combinable therapeutic agents are nitrates or NO donors such as isosorbide mononitrate, isosorbide dinitrate or molsidomine.

Various administration routes can be utilized for the use according to the invention of said compounds for treating said pathological conditions, for example oral, sublingual, buccal, generally via the oral mucosa, nasal, inhalational, rectal, transdermal or in a narrower sense parenteral. Parenteral administration can take place with avoidance of an absorption step (e.g. intravenous, intraarterial, intracardiac, intraspinal or intralumbal) or with inclusion of absorption (e.g. intramuscular, subcutaneous, intracutaneous, percutaneous or intraperitoneal). Administration as implant is also possible. Oral, intravenous and inhalational administration are preferred.

The present invention further relates to medicaments which comprise at least one of the compounds used according to the invention, normally together with one or more inert, non-toxic, pharmaceutically suitable excipients.

Administration forms suitable for oral administration are those which function according to the state of the art and deliver the compounds of the invention in a rapid and/or modified way, and which contain the compounds of the invention in crystalline and/or amorphous and/or dissolved form. These include coated or uncoated tablets, hard capsules made of gelatin, HPMC, pullulan or other materials, soft gelatin capsules, chewable tablets, effervescent tablets, tablets for preparing a solution or suspension, orally disintegrating pharmaceutical forms such as tablets, films or lyophilizate flakes, powders, granules, pellets, chewing gums, solutions, emulsions, suspensions and semisolid oral pharmaceutical forms such as gels. Controlled-release formulations are suitable for the administration of compounds of the invention, especially those with a short half-life. These include for example matrix tablets, erosion matrix tablets, tablets or minitablets with diffusion-controlling or gastro-resistant coating, capsules with pellets, granules or tablets with diffusion-controlling or gastro-resistant coating, capsules with eroding pellets, granules or tablets, osmotic-release tablets. Compounds of short half-life are those whose half-life is less than 8 hours after administration. An accurate description of controlled-release formulations and the manufacture thereof is provided in the section sustained-release pharmaceutical forms for controlled release of one of the mentioned compounds of the invention and a further therapeutic agent (see below).

Administration forms suitable for parenteral administration are, inter alia, injection and infusion preparations in the form of solutions, suspensions, emulsions, lyophilisates or sterile powders. Needleless injection of the compounds of the invention is also possible.

Solutions or suspensions for use in nebulizers, capsules filled with powder or powders for use in inhalers and metered aerosols, for example compressed gas packs with suspensions or solutions for inhalation are suitable for inhalational administration of the compounds of the invention.

Suitable for other administration routes are for nasal drops, solutions or sprays, tablets for lingual, sublingual or buccal administration, films or capsules, suppositories for rectal use or enemas, transdermal therapeutic systems (such as, for example, patches) and implants or stents.

The compounds used according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and flavorings and/or odorizers.

Dosage forms suitable for processing one of said compounds and a further therapeutic agent to give a joint oral dosage form (fixed combination) are all those detailed above. If there is mutual impairment of stability, the combination partners in the pharmaceutical form can be spatially separated. To this end, for example separate granules or pellets are packed into capsules, or two granules are compressed to give a monolayer or bilayer tablet. It is also possible in addition to coat at least one of the combination partners or a powder, granules or pellet preparation of at least one combination partner with polymer and subsequently to process further to a tablet or capsule.

In certain cases of said combinations, a delayed, controlled release of said compounds of the invention, or of the further therapeutic agent(s) or of all components may be particularly advantageous. This may lead to an improved pharmacological effect, reduce the occurrence of unwanted side effects or contribute to simplifying the therapy for the patient. The invention therefore also relates to formulations which comprise one of said compounds of the invention and one of said further therapeutic agents and deliver both ingredients or one of the two ingredients in controlled form. It is moreover possible for both the compound of the invention and the further therapeutic agent also to be delivered only partly in controlled form, while the other portion is released rapidly. Combinations preferred in this connection are those in which the compound of the invention and/or the further therapeutic agent have a half-life of less than 8 hours and are present in controlled-release formulation. Very particularly preferred combinations in this connection are those with controlled-release formulation of vardenafil, sildenafil, furosemide, piretanide, metoprolol, pindolol, propranolol, nifedipine, nisoldipine, isradipine, felodipine, nilvadpipine, nitrendipine, quinidine bisulfate, disopyramide phosphate, isosorbide mononitrate and isosorbide dinitrate.

Controlled-release formulations mean in this connection those which deliver the active ingredient with an average release rate of 80% in more than 45 minutes. Particularly preferred pharmaceutical formulations are those which deliver the active ingredient in a controlled way with an average release rate of between 80% in 2 hours and 80% in 16 hours. To ascertain the average release rate, the pharmaceutical formulations of the present invention are tested in “apparatus 2” of the USP (The United States Pharmacopeia). A test medium used is 900 ml of phosphate buffer with pH 6.8 and with addition of 0.1% sodiumlaurylsulfate. The speed of rotation of the stirrer is 75 revolutions per minute. Samples are taken through an 8 μm filter and their active ingredient content is determined. The amount of active ingredient determined as dissolved in this way is converted into percent by weight of the amount of active ingredient employed.

For controlled release of one of said compounds of the invention and a further therapeutic agent, two of the sustained-release pharmaceutical forms are used. These represent a combination of a controlled-release formulation of one of said compounds of the invention, for example vardenafil, with a controlled-release formulation of a further therapeutic agent, for example metoprolol or furosemide. Examples suitable for this purpose are capsules which comprise two types of pellets, minitablets or tablets, specifically those with controlled release of one of said compounds of the invention and those with controlled release of the further therapeutic agent. Sustained-release tablets which consist of at least two different active ingredient layers represent a further possibility. One of them releases one of said compounds of the invention in a controlled way, and another the further therapeutic agent. Possible besides these are osmotic-release tablets. These comprise both combination partners in the active ingredient layer. Also optionally possible is an osmotically active swelling layer free of active ingredient. The resulting mono- or bilayer tablet is coated with a water-insoluble but permeable coating, for example of cellulose acetate, and provided with at least one orifice on the active ingredient-containing side to release the active ingredient.

If controlled release is intended for only one of the combination partners (one of said compounds of the invention or the further therapeutic agent), singly sustained-release pharmaceutical forms are used. Singly sustained-release pharmaceutical forms are for example capsules which comprise the controlled-release combination partner in the form of pellets, minitablets or tablets and the fast-release combination partner in the form of a powder, compact, tablet or pellet. Sustained-release tablets which consist of at least two different active ingredient layers represent a further possibility. One comprises the controlled-release combination partner. Its release is controlled for example by incorporation into an insoluble matrix or erodible matrix. Another active ingredient layer of the tablet comprises the non-controlled-release combination partner in conventional fast-release form. Sustained-release tablets with a monolayer structure represent a further possibility. These comprise the controlled-release combination partner in the form of diffusion pellets and those not for sustained release. Besides these, osmotic-release tablets are possible. These have for example a trilayer structure and comprise a first layer with a non-controlled-release combination partner, a second layer with the controlled-release combination partner and an osmotically active swelling layer free of active ingredient. The resulting trilayer tablet is coated with a water-insoluble but permeable coating, for example of cellulose acetate, and provided with at least one orifice on the active ingredient-containing side to release the active ingredient.

Particularly suitable for formulating controlled-release preparations of the compound of the invention or of a further therapeutic agent for subsequent packing into a capsule are diffusion-controlled pellets. Diffusion-controlled pellets are produced for example by layering neutral pellets of sucrose or microcrystalline cellulose with a mixture of the active ingredient, conventional binders, if necessary an acid and further conventional excipients, and then coating with a diffusion coating which may comprise a plasticizer. Binders preferably used are hydroxypropylmethyl-cellulose or polyvinylpyrrolidone. It is likewise possible to employ other natural, synthetic or semisynthetic polymers such as, for example, methylcellulose, hydroxypropylcellulose, sodium-carboxymethylcellulose, polyacrylic acids, polyvinyl alcohols or gelatin. Particularly suitable as diffusion coating is ethylcellulose as is commercially available for example as aqueous dispersion under the name Aquacoat® or Surelease®. However, other materials such as poly[(methacrylic acid) (ethyl acrylate)] (1:1) or other acrylates (Eudragit®), cellulose acetate or cellulose acetate butyrate can also be used. Examples of suitable plasticizers are phthalic acid derivatives (e.g. dimethyl phthalate, diethyl phthalate, dibutyl phthalate), citric acid derivatives (e.g. tri ethyl citrate, tributyl citrate, acetyl triethyl citrate), other esters (e.g. diethyl sebacate, triacetin), fatty acids and derivatives (glycerol monostearate, acetylated fatty acid glycerides, castor oil or other natural oils, Miglyol), polyols (glycerol, 1,2-propanediol, polyethylene glycol of varying chain lengths). The nature and amount of the plasticizer are moreover adjusted to achieve the above-defined release according to the invention and the necessary stability of the pellets. Adjustment of the above-defined release further takes place by controlling the pore size of the diffusion coating and/or its thickness. Pore formers which can be employed to control the pore size are all appropriate soluble polymers such as, for example, polyethylene glycols, polyvinylpyrrolidones, hydroxypropyl-methylcelluloses, carboxymethylcelluloses or salts thereof, methylcelluloses, dextrins, maltodextrins, cyclodextrins, dextrans or other soluble compounds such as, for example, salts (sodium chloride, potassium chloride, ammonium chloride etc.), urea, sugars (glucose, sucrose, fructose, lactose etc.), sugar alcohols (mannitol, sorbitol, lactitol etc.). The proportion of pore former in the amount of coating is in this case from 0 to 50% (W/W) (W=mass). It is particularly important in the case of pellets to use a particular weight ratio of active ingredient-coated pellets to the diffusion membrane, and a particular ratio of diffusion coating to amount of plasticizer. Parts of the plasticizer employed may evaporate during the coating and subsequent thermal treatment. If the limiting conditions are altered, the amount of diffusion coating applied must be changed. Thus, for example, it is necessary to apply a larger amount if the desired release rate is reduced, the amount of pore formers is increased or, with certain plasticizers, the proportion of plasticizer is reduced. It is necessary to apply a smaller amount if the desired release rate is increased, the amount of pore formers is reduced or, with certain plasticizers, the proportion of plasticizer is increased. The diffusion pellets can be produced for example by suspending or dissolving the active ingredient in water and thickening with a concentrated hydroxypropylmethylcellulose solution. The suspension or solution obtained in this way is absorbed on neutral pellets in a spraying process in a fluidized bed system. The pellets are then coated with a diffusion membrane, preferably in a fluidized bed system by spraying on, for example an aqueous ethylcellulose dispersion or organic ethylcellulose solution which comprises a suitable, physiologically tolerated plasticizer. The pellets are then thermally treated at temperatures of from 50 to 125° C., preferably 60 to 110° C. In this connection, higher temperatures in the thermal treatment lead to smaller amounts of applied coating tending to be sufficient to achieve the release according to the invention, and the resulting pellets being more physically stable on storage. The thickness of the diffusion membrane, type of plasticizer, amount of plasticizer and pellet size are chosen so that the resulting release rate of the active ingredient is 80% in more than 45 minutes, preferably between 80% in 2 hours to 16 hours. The amount of pellets corresponding to a daily dose is packed into a hard gelatin capsule. Besides the described coating of neutral pellets, other methods of pellet production are also feasible, such as wet extrusion and rounding, rotor granulation, fluidized bed agglomeration or thermal extrusion. It is alternatively possible also to produce minitablets with a diameter of 1-4 mm. The active ingredient-containing pellets or minitablets are subsequently coated with a diffusion membrane as described.

Suitable in another embodiment of the pharmaceutical formulation of the invention for formulating the controlled-release preparations of the compound of the invention or of a further therapeutic agent for subsequent packing into a capsule are tablets which comprise the active ingredient in a matrix of a water-swellable polymer. The size of these tablets is such that there is space for one or more tablets inside the capsule. The tablets can be packed in uncoated form into the capsule or previously be provided with a coating, for example a coating insoluble in gastric juices.

Tablets for subsequent packing into a capsule which comprise the active ingredient in a matrix of a water-swellable polymer are produced as follows. These so-called matrix formulations expediently comprise from 0.1 to 70% by weight, preferably 0.2 to 60% by weight, of the active ingredient. The proportionate amount of the matrix of the water-swellable polymer is expediently from 10 to 95% by weight, preferably 20 to 60% by weight. Pharmaceutical preparations according to the invention in the form of erodible tablets are particularly preferred. These tablets are characterized in that, besides conventional excipients and carriers, as well as tableting excipients, they comprise a defined amount of water-swellable, hydrogel-forming polymers, where these polymers must have a viscosity of at least 15, preferably at least 50 cps (measured as 2% strength aqueous solution at 20° C.). Examples of conventional excipients and carriers are lactose, microcrystalline cellulose, mannitol or calcium phosphates. Examples of conventional tableting aids are magnesium stearate, talc or colloidal silicon dioxide (Aerosil®). These are present expediently in an amount of from 0.5 to 3% by weight in the case of magnesium stearate, and expediently in an amount of from 0.1 to 1% by weight in the case of colloidal silicon dioxide. Water-soluble, hydrogel-forming polymers which are preferably employed are hydroxypropylcelluloses, hydroxypropylmethyl-celluloses (HPMC), methylcelluloses, carboxymethylcellulose, alginates, galactomannans, polyacrylic acids, polymethacrylic acids or copolymers of methacrylic acid and methyl methacrylate, guar, agar, pectin, tragacanth, gum arabic, xanthan and mixtures of these substances. The use of HPMC is particularly preferred. In this case, the erodible tablets according to the invention should preferably comprise at least 10% by weight, based on the mass of a tablet, of a hydroxypropylmethylcellulose type whose viscosity (measured as 2% strength aqueous solution at 20° C.) is at least 15, preferably at least 50 cps. The pharmaceutical formulation which includes the active ingredient in a matrix of a water-swellable polymer is produced by mixing the active ingredient, the polymer and suitable excipients and carriers (as described above), conventional tableting aids (as described above), and tableting directly. It is further possible to granulate the active ingredient, the water-swellable polymer and suitable carriers in a fluidized bed. In this case, the amount and viscosity of the water-swellable polymer is chosen so that the resulting tablets have the average release rates described above for the compound of the invention or a further therapeutic agent. The dry granules are screened, mixed with a lubricant such as, for example magnesium stearate and tableted. The tablet is then coated where appropriate. Erodible tablets with a diameter of from 3 mm to 7 mm are preferred for subsequent packing into a capsule.

The combination partner not for sustained release can be introduced in the form of a powder, granules, pellet or tablet into the capsule. Conventional fast-release formulations are suitable for this purpose.

In a further embodiment of the pharmaceutical formulation, the combination partners are present in a bilayer tablet. This consists of two controlled-release layers or of a controlled- and of a fast-release layer.

Formulation of each controlled-release layer is based on the principles set forth above for the matrix formulation for subsequent packing into a capsule. To formulate each fast-release layer, the active ingredient is mixed with suitable excipients and carriers (as described above) and conventional tableting aids (as described above) and tableted directly. It is further possible to granulate the active ingredient and suitable carriers in a fluidized bed, in a mixing granulator or in a roll compactor. The dry granules are screened, mixed with a lubricant such as, for example, magnesium stearate, and tableted. Suitable for the tableting is in particular a bilayer press provided with two charging and compression stations. The tablet is then coated where appropriate. In order to prevent the initial release rate of one of the combination partners being too high, the bilayer tablet may also be provided with a third layer free of active ingredient.

In a further embodiment of the pharmaceutical formulation, the combination partners are present in a monolayer tablet. This comprises one combination partner in controlled-release formulation and the other combination partner in fast-release form. The diffusion-controlled pellets described above are particularly suitable as controlled-release formulation for subsequent incorporation into a monolayer tablet. They are mixed with the active ingredient which is to be combined in fast-release form and with further excipients, carriers and tableting aids and compressed to a monolayer tablet. Granulation of the fast-release excipient and subsequent coating of the tablet are also possible.

A further embodiment of the pharmaceutical formulation of the present invention is an osmotic pharmaceutical release system. Such osmotic pharmaceutical release systems are in principle known in the state of the art and are dealt with in detail for example in Richard W. Baker, “Osmotic Drug Delivery: A Review of the Patent Literature”, Journal of Controlled Release 35 (1995) 1-21. The pharmaceutical formulation as osmotic pharmaceutical release system preferably consists of
a) a core which comprises the active ingredients, where appropriate a hydrophilic polymeric swelling agent and where appropriate a water-soluble substance to induce osmosis, and
b) a shell which is permeable by water and impermeable by the components of the active ingredient-containing core
c) an aperture through the shell b) for transporting the ingredients present in the core into the surrounding body fluid.

This specific osmotic pharmaceutical release system is described in principle in the state of the art, for example in DE-A-2 328 409 or U.S. Pat. No. A-385,770. Concerning the materials for the shell, reference may be made to EP-A-0 277 092, and U.S. Pat. No. A-3,916,899 and U.S. Pat. No. A-3,977,404 which are mentioned therein.

Concerning suitable hydrophilic polymeric swelling agents, reference may be made for example to the polymeric swelling agents mentioned in the EP-A-0 277 092 and WO 96/40080. It is possible to use for example ethylene oxide homopolymers (polyethylene glycol) with various degrees of polymerization, which are known for example under the name Polyox, having molecular weights of between 100 000 to 8 000 000, and vinylpyrrolidone-vinyl acetate copolymers, and further water-swellable polymers mentioned in U.S. Pat. No. A-3,865,108, U.S. Pat. No. A-4,002,173 and U.S. Pat. No. A-4,207,893. Water-soluble substances for inducing osmosis are in principle all water-soluble substances whose use is acceptable in pharmacy and which are mentioned for example in the pharmacopeias or in “Hager's Handbuch der Pharmazeutischen Praxis, 1990-1995, Springer Verlag” and Remington's Pharmaceutical Sciences as water-soluble excipients. Specific water-soluble substances are salts of inorganic or organic acids or nonionic organic substances with high water solubility such as, for example, carbohydrates such as sugars etc. Production of an orifice in the shell of the tablet is known in the state of the art and described for example in U.S. Pat. Nos. 3,485,770 and 3,916,899. The release rate is adjusted through the type and amount of the semipermeable material forming the shell, by the type and amount of the hydrophilic polymeric swelling agent which is present where appropriate, and of the water-soluble substance which is present where appropriate to induce osmosis. The combination partners of the present invention can be introduced in various ways into an osmotic pharmaceutical release system. For controlled delivery of both active ingredients, they are mixed with the excipients and compressed together in one active ingredient layer. If only one combination partner is to undergo controlled release, this can either be introduced separately into the coated shell of the tablet, or the active ingredient which is not to undergo controlled release is compressed to a separate active ingredient layer which is pumped out of the pharmaceutical release system first, before the combination partner which is to undergo controlled release.

It has generally proved advantageous on parenteral administration to administer amounts of the compound of the invention of about 0.001 to 10 mg/kg, preferably about 0.01 to 1 mg/kg, of body weight to achieve effective results. The amount on oral administration is about 0.01 to 100 mg/kg, preferably about 0.1 to 30 mg/kg, and very particularly preferably 0.1 to 10 mg/kg, of body weight. In every case the dose may be carried out inclusively.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

### EXAMPLES

The following exemplary embodiments explain the invention. The invention is not restricted to the examples.

### Example 1

2-[2-Ethoxy-5-(4-methylpiperazine-1-sulfonyl)phenyl]-5-methyl-7-propyl-3H-imidazo[5,1-f][1,2,4]triazin-4-one is prepared in accordance with example 16 in WO 99/24433. The compound is micronized to an average particle size of 3.8 μm. Per tablet, 5 mg of this powder are mixed with 80 mg of microcrystalline cellulose and 6 mg of croscarmellose sodium, dry granulated and then mixed with 1 mg of magnesium stearate and 0.5 mg of colloidal silicon dioxide and compressed to tablets of 92.5 mg. The tablets are employed for the treatment of pulmonary hypertension.

### Example 2

2-[2-Ethoxy-5-(4-ethylpiperazine-1-sulfonyl)phenyl]-5-methyl-7-propyl-3H-imidazol[5,1-f][1,2,4]triazin-4-one hydrochloride trihydrate is prepared in accordance with example 336 in WO 99/24433. The powder is micronized and mixed with five times the amount of microcrystalline cellulose. 142 mg of this mixture are packed into hard gelatin capsules. The capsules are blister-packed and packaged together with a package leaflet in an outer box. It is stated on the package leaflet that the capsules are used for treating altitude sickness.

### Example 3

| Vardenafil hydrochloride trihydrate | 119 g | |
|---|---|---|
| Sodium chloride | 9.00 | kg |
| Lactic acid solution 20% | 5.00 | kg |
| 2M sodium hydroxide solution at pH 4.0 | 0 to 10 | kg |
| Water for injections | ad total amount employed | |
| Total amount | 1 005.1 | kg |

The solution is sterilized by filtration, dispensed in 50 ml portions into infusion bottles, closed with an infusion stopper, crimp-capped and sterilized in the final container at 121° C. for 15 minutes. The infusion is administered slowly over 12 hours using an infusion pump to patients with portopulmonary hypertension.

### Example 4

10 kg of micronized tadalafil is mixed with 40 kg of lactose monohydrate and packed in 25 mg portions into capsules. The capsules are inserted into a powder inhaler and inhaled by patients with COPD.

### Example 5

A bilayer tablet is produced in a direct tableting process as follows: 20.85 kg of telmisartan sodium salt and 174.15 kg of spray-dried mannitol are mixed, sieved and, after addition of 5 kg of magnesium stearate, mixed again. The mixture is used for layer A of the bilayer tablet. 10 kg of micronized vardenafil, 64.6 kg of spray-dried mannitol and 100 kg of microcrystalline cellulose are mixed, sieved and, after addition of 5.4 kg of magnesium stearate, mixed again. The mixture is used for layer B of the bilayer tablet. The two mixtures are compressed with the aid of a bilayer tablet press to round bilayer tablets with a diameter of 11 mm which consist of 200 mg of layer A (equivalent to 20 mg of telmisartan) and 180 mg of layer B (equivalent to 10 mg of vardenafil). The bilayer tablets are packed into HDPE bottles and provided with a package leaflet stating that the bilayer tablet is used for the treatment of pulmonary hypertension, COPD and bronchial asthma.

### Example 6

A bilayer tablet is produced in a drum granulation process as follows: 32 kg of propranolol hydrochloride, 40.8 kg of microcrystalline cellulose and 40 kg of lactosemonohydrate are mixed, sieved, drum-granulated dry and, after addition of 0.8 kg of colloidal silicon dioxide, 4.8 kg of talc and 1.6 kg of magnesium stearate, mixed again. The mixture is used for layer A of the bilayer tablet. 18.964 kg of vardenafil hydrochloride trihydrate, 87.836 kg of microcrystalline cellulose, 48 kg of hydroxypropylmethylcellulose of the USP 2208 type and viscosity level 100 cP are mixed, sieved, drum-granulated dry and, after addition of 1.6 kg of colloidal silicon dioxide and 3.6 kg of magnesium stearate, mixed again. The two mixtures are compressed with a bilayer tablet press to round bilayer tablets with a diameter of 10 mm, which consists of 150 mg of layer A (equivalent to 40 mg of propranolol hydrochloride) and 200 mg of layer B (equivalent to 20 mg of vardenafil). The bilayer tablets are used for treating right heart failure, left heart failure and global failure.

The PDE- and PDE 5-inhibiting effect of the compounds used according to the invention can be determined as follows:

### PDE 5 Inhibition Assay

The inhibitory effect is assayed by using the phosphodiesterase [³H] cGMP-SPA enzyme assay supplied by Amersham Life Science. The assay is carried out in accordance with the experimental protocol indicated by the manufacturer. Human recombinant PDE 5 which has been expressed in a bacculovirus system is used. The substance concentration at which the reaction rate is reduced by 50% is measured.

Exemplary embodiments 1 and 2 show IC₅₀ values respectively of 0.6 and 0.7 nM in this assay.

### PDE Inhibition Assays

Recombinant PDE1C (GenBank/EMBL Accession Number: NM_{—}005020, Loughney et al. J. Biol. Chem. 1996, 271, 796-806), PDE2A (GenBank/EMBL Accession Number: NM_{—}002599, Rosman et al. Gene 1997, 191, 89-95), PDE3B (GenBank/EMBL Accession Number: NM_{—}000922, Miki et al. Genomics 1996, 36, 476-485), PDE4B (GenBank/EMBL Accession Number: NM_{—}002600, Obernolte et al. Gene. 1993, 129, 239-247), PDE5A (GenBank/EMBL Accession Number: NM_{—}001083, Loughney et al. Gene 1998, 216, 139-147), PDE7B (GenBank/EMBL Accession Number: NM_{—}018945, Hetman et al. Proc. Natl. Acad. Sci. U.S.A. 2000, 97, 472-476), PDE8A (GenBank/EMBL Accession Number: AF_{—}056490, Fisher et al. Biochem. Biophys. Res. Commun. 1998, 246, 570-577), PDE9A (Fisher et al., J. Biol. Chem., 1998, 273 (25): 15559-15564), PDE10A (GenBank/EMBL Accession Number: NM_{—}06661, Fujishige et al. J Biol. Chem. 1999, 274, 18438-45), PDE11A (GenBank/EMBL Accession Number: NM_{—}016953, Fawcett et al. Proc. Natl. Acad. Sci. 2000, 97, 3702-3707) were expressed in Sf9 cells with the aid of the pFASTBAC baculovirus expression system (GibcoBRL).

The test substances are dissolved in 100% DMSO and serially diluted to determine their in vitro effect on PDE9A. Typically, serial dilutions from 200 μM to 1.6 μM are prepared (resulting final concentrations in the assay: 4 μM to 0.032 μM). 2 μL portions of the diluted substance solutions are introduced into the wells of microtiter plates (Isoplate; Wallac Inc., Atlanta, Ga.). Then 50 μL of a dilution of the PDE9A preparation described above are added. The dilution of the PDE9A preparation is chosen so that less than 70% of the substrate is converted during the subsequent incubation (typical dilution: 1:10 000; dilution buffer: 50 mM Tris/HCl pH 7.5, 8.3 mM MgCl₂, 1.7 mM EDTA, 0.2% BSA). The substrate, [8-³H] guanosine 3′,5′-cyclic phosphate (1 μCi/μL; Amersham Pharmacia Biotech., Piscataway, N.J.) is diluted 1:2000 with assay buffer (50 mM Tris/HCl pH 7.5, 8.3 mM MgCl₂, 1.7 mM EDTA) to a concentration of 0.0005 μCi/μL. The enzyme reaction is finally started by adding 50 μL (0.025 μCi) of the diluted substrate. The assay mixtures are incubated at room temperature for 60 min and the reaction is stopped by adding 25 μl of a PDE9A inhibitor (e.g. the compound from example 1 in WO/2004/026286, final concentration 10 μM) dissolved in assay buffer. Immediately thereafter, 25 μL of a suspension containing 18 mg/mL Yttrium Scintillation Proximity Beads (Amersham Pharmacia Biotech., Piscataway, N.J.) are added. The microtiter plates are sealed with a film and left to stand at room temperature for 60 min. The plates are then measured for 30 s per well in a Microbeta scintillation counter (Wallac Inc., Atlanta, Ga.). IC₅₀ values are determined from the graphical plot of the substance concentration versus the percentage inhibition.

The in vitro effect of test substances on recombinant PDE3B, PDE4B, PDE7B, PDE8A, PDE10A and PDE11A is determined in accordance with the assay protocol described above for PDE9A with the following adaptations: [5′,8-³H] adenosine 3′,5′-cyclic phosphate (1 μCi/μL; Amersham Pharmacia Biotech., Piscataway, N.J.) is used as substrate. Addition of an inhibitor solution to stop the reaction is unnecessary. Instead, the incubation of substrate and PDE is followed immediately by addition of the yttrium scintillation proximity beads as described above and thus the reaction is stopped. To determine a corresponding effect on recombinant PDE1C, PDE2A and PDE5A, the protocol is additionally adapted as follows: with PDE1C, additionally 10⁻⁷M calmodulin and 3 mM CaCl₂ are added to the reaction mixture. PDE2A is stimulated in the assay by adding 1 μM cGMP and is assayed with a BSA concentration of 0.01%. The substrate employed for PDE1C and PDE2A is [5′,8-³H] adenosine 3′,5′-cyclic phosphate (1 μCi/μL; Amersham Pharmacia Biotech., Piscataway, N.J.), and for PDE5A is [8-³H] guanosine 3′,5′-cyclic phosphate (1 μCi/μL; Amersham Pharmacia Biotech., Piscataway, N.J.).

1. The use of PDE 5 inhibitors for manufacturing a medicament for the treatment of: primary pulmonary hypertension, secondary pulmonary hypertension, pulmonary arterial hypertension, portopulmonary hypertension, hepatopulmonary syndrome, pulmonary hypertension caused by medicaments (amphetamines), interstitial lung disease, pulmonary hypertension occurring with HIV, thromboembolic pulmonary hypertension, pulmonary hypertension in children and neonates, pulmonary hypertension induced by atmospheric hypoxia (altitude sickness), COPD, emphysema, chronic bronchial asthma, mucoviscidosis-related pulmonary hypertension, right-heart failure, left-heart failure and global failure.

2. The use of PDE 5 inhibitors for manufacturing a medicament for the treatment of: isolated systolic hypertension (ISH) and hardening of blood vessels.

3. The use as claimed in claim 1 or 2 of compounds of the formula (I) 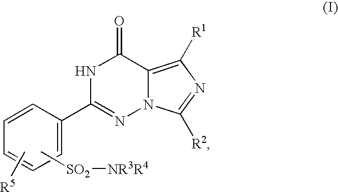
in which
R¹ is methyl or ethyl,
R² is ethyl or propyl,
R³ and R⁴ are identical or different and are a straight-chain or branched alkyl chain having up to 5 carbon atoms which is optionally substituted up to twice identically or differently by hydroxy or methoxy,
or
R³ and R⁴ together with the nitrogen atom form a piperidinyl, morpholinyl, thiomorpholinyl ring or a radical of the formula 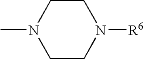
in which
R⁶ is hydrogen, formyl, acyl or alkoxycarbonyl having in each case up to 3 carbon atoms,
or
is straight-chain or branched alkyl having up to 3 carbon atoms which is optionally substituted once to twice, identically or differently, by hydroxy, carboxyl, straight-chain or branched alkoxy or alkoxycarbonyl having in each case up to 3 carbon atoms or by groups of the formulae —(CO)_{f}—NR⁷R⁸ or —P(O)(OR⁹)(OR¹⁰),
in which
f is a number 0 or 1,
R⁷ and R⁸ are identical or different and are hydrogen or methyl,
R⁹ and R¹⁰ are identical or different and are hydrogen, methyl or ethyl,
or
R⁶ is cyclopentyl,
and the heterocycles mentioned under R³ and R⁴ and formed together with the nitrogen atom are optionally substituted once to twice, identically or differently, optionally also geminally, by hydroxy, formyl, carboxyl, acyl or alkoxycarbonyl having in each case up to 3 carbon atoms or groups of the formulae —P(O)(OR¹¹)(OR¹²) or —(Co)ᵢ—NR¹³R¹⁴,
in which
R¹¹ and R¹² are identical or different and are hydrogen, methyl or ethyl,
i is a number 0 or 1,
and
R¹³ and R¹⁴ are identical or different and are hydrogen or methyl,
and/or the heterocycles mentioned under R³ and R⁴ and formed together with the nitrogen atom are optionally substituted by straight-chain or branched alkyl having up to 3 carbon atoms, which is optionally substituted once to twice, identically or differently, by hydroxy, carboxyl or by a radical of the formula —P(O)OR¹⁵R¹⁶,
in which
R¹⁵ and R¹⁶ are identical or different and are hydrogen, methyl or ethyl,
and/or the heterocycles mentioned under R³ and R⁴ and formed together with the nitrogen atom are optionally substituted by N-linked piperidinyl or pyrrolidinyl,
and
R⁵ is ethoxy or propoxy,
and the salts and solvates thereof and the solvates of the salts.

4. The use as claimed in claim 1 or 2 of compounds of the formula (Ia) 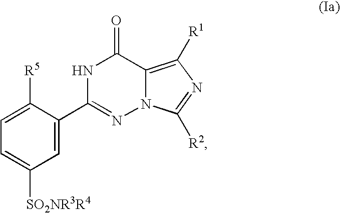
in which R¹, R², R³, R⁴ and R⁵ each have the meaning indicated in claim 2,
and the salts and solvates thereof and the solvates of the salts.

5. The use as claimed in claim 1 or 2 of compounds characterized in that they are selected from the group having the following structures:
Structure

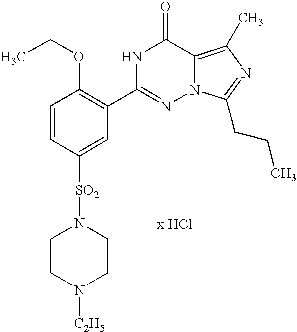

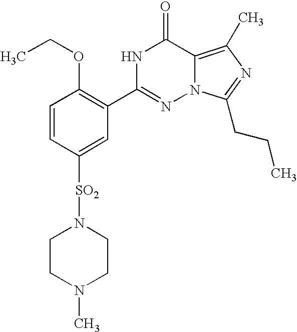

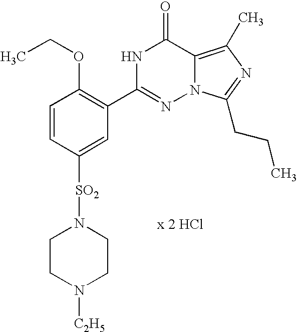

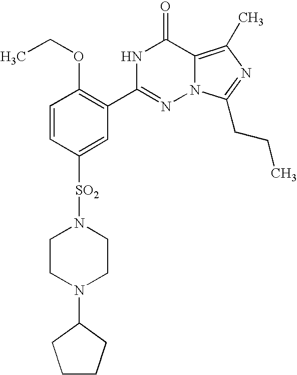

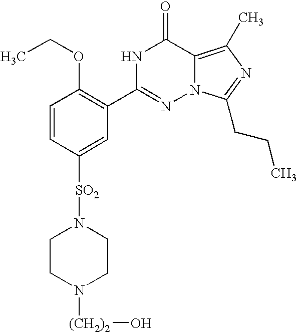

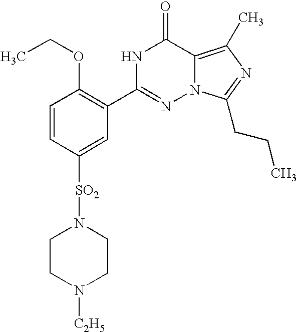

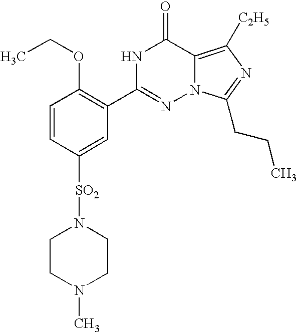

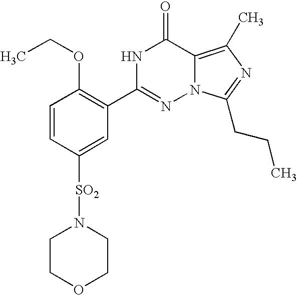

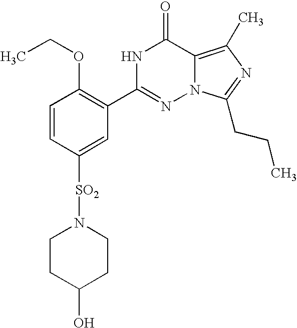

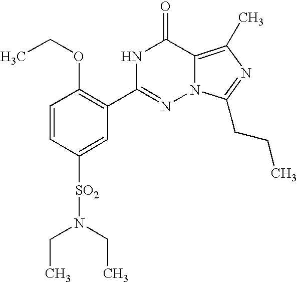

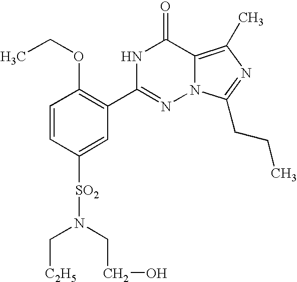

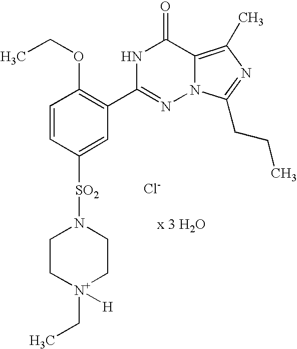

6. The use of sildenafil and the salts thereof as claimed in claim 2.

7. The use of tadalafil as claimed in claim 2.

8. The use of PDE 5 inhibitors, especially of 2-phenyl-substituted imidazotriazinone derivatives and very especially of compounds as claimed in claim 5, 6 and 7 in combination with one or more therapeutic agents for the treatment of: primary pulmonary hypertension, secondary pulmonary hypertension, pulmonary arterial hypertension, portopulmonary hypertension, hepatopulmonary syndrome, pulmonary hypertension caused by medicaments (amphetamines), interstitial lung disease, pulmonary hypertension occurring with HIV, thromboembolic pulmonary hypertension, pulmonary hypertension in children and neonates, pulmonary hypertension induced by atmospheric hypoxia (altitude sickness), COPD, emphysema, chronic bronchial asthma, mucoviscidosis-related pulmonary hypertension, right-heart failure, left-heart failure and global failure.

9. The use of PDE 5 inhibitors, especially of compounds as claimed in claim 5, 6, and 7 in combination with one or more therapeutic agents for the treatment of isolated systolic hypertension (ISH) and hardening of blood vessels.

10. The use of the compounds as claimed in claim 8 or 9 characterized in that the further therapeutic agent(s) are selected from:
oxygen or NO inhalation, it being possible for the inhalation also to be in gradually increasing or decreasing doses and pulsatile,
administration of diuretics, antiarrhythmics, calcium channel blockers, vasodilators
inhalational, oral, subcutaneous, transdermal or intravenous administration of prostanoids such as, for example, PGI2 and derivatives, prostacycline and its analogs, of endothelin receptor antagonists, intravenous or inhalational administration of adrenomedullin,
administration of anticoagulants, phosphodiesterase inhibitors, especially PDE3 inhibitors, PDE4 inhibitors, PDE5 inhibitors, especially from WO 2004022557, WO 2004019945, WO 2004018457, WO 2004018450, WO 2004018449, WO 2004017974, WO 2003074055, WO 2003070279, WO 2002085906, WO 2002085885, WO 2004018465, sildenafil, tadalafil, milrinone
administration of cardiac glycosides
administration of beta-receptor blocker
administration of alpha-receptor blocker
administration of ACE inhibitors
administration of angiotensin II receptor antagonists
administration of nitrates or molsidomine

11. The administration of compounds as claimed in claim 1 or 2 using the oral administration route.

12. The administration of compounds as claimed in claim 1 or 2 using the intravenous administration route.

13. The administration of compounds as claimed in claim 1 or 2 using the inhalational administration route.

14. The administration of combinations as claimed in any of claims 8-10, characterized in that the combination partners are administered by a route selected from:
oral, intravenous, inhalational,
where the route of the individual combination partners may be identical or different.

15. A medicament comprising compounds as defined in claims 3 to 7, characterized in that it comprises a coated or uncoated tablet, a hard capsule, a soft gelatin capsule, chewable tablet, effervescent tablet, tablet for preparing a solution or suspension, an orally disintegrating pharmaceutical form, powder, granules, pellets, chewing gum, solution, suspension or gel for oral use, solution for injection, solution for infusion, lyophilizate, sterile powder, solution or suspension for inhalation for use in nebulizers, capsule or powder for inhalation or metered aerosol.

16. A medicament comprising compounds as defined in claims 3 to 7, characterized in that it is a controlled-release pharmaceutical form.

17. A medicament comprising combinations as defined in claims 8 to 10, characterized in that it comprises a coated or uncoated tablet, a hard capsule, a soft gelatin capsule, chewable tablet, effervescent tablet, tablet for preparing a solution or suspension, an orally disintegrating pharmaceutical form, powder, granules, pellets, chewing gum, solution, suspension or gel for oral use, solution for injection, solution for infusion, lyophilizate, sterile powder, solution or suspension for inhalation for use in nebulizers, capsule or powder for inhalation or metered aerosol.

18. A medicament as claimed in claim 17, where the ingredients of the combination are spatially separated from one another in the pharmaceutical form.

19. A medicament comprising combinations as defined in claims 8 to 10, characterized in that it is a pharmaceutical form which comprises two combination partners, where at least one of the combination partners undergoes controlled release.
